(19) United States
(12) Patent Application Publication
(10) Pub. No.: US 2022/0030855 A1
(43) Pub. Date: Feb. 3, 2022

(54) FUNCTIONALIZED SET OF CARBON ATOMS IN THE SP2 HYBRIDIZATION STATE, A METHOD FOR PREPARING IT AND ITS USES, IN PARTICULAR FOR RENDERING A SURFACE ANTIBACTERIAL

(71) Applicant: Commissariat a I'Energie Atomique et aux Energies Alternatives, Paris (FR)

(72) Inventors: Thomas Alava, Grenoble (FR); Madhav Kumar, Grenoble (FR)

(73) Assignee: Commissariat a I'Energie Atomique et aux Energies Alternatives, Paris (FR)

(21) Appl. No.: 17/383,817

(22) Filed: Jul. 23, 2021

(30) Foreign Application Priority Data
Jul. 29, 2020 (FR) 2008040

(51) Int. Cl. A01N 25/24; A01N 63/50; A01N 25/08; A01N 25/10; A01P 1/00

(52) U.S. Cl. CPC A01N 25/24; A01N 63/50; A01P 1/00; A01N 25/10; A01N 25/08

(57) ABSTRACT

The present invention relates to a set of carbon atoms in the sp² hybridization state functionalized with an antibacterial antibody or a lectin, and to an antibacterial peptide, a method for preparing it, and its uses, in particular for rendering a surface antibacterial. The present invention is also directed to a material covered with such a functionalized set, as well as to a method for preparing it.

The present invention relates to a set of carbon atoms in the sp² hybridization state functionalized with an antibacterial antibody or a lectin, and to an antibacterial peptide, a method for preparing it, and its uses, in particular for rendering a surface antibacterial. The present invention is also directed to a material covered with such a functionalized set, as well as to a method for preparing it.

In addition to the so-called “disinfectants” products, and the uses one makes of them to clean or even sterilize the floors, the hands, and various surfaces, there is another approach to avoid the bacterial development, consisting in rendering the surfaces directly antibacterial. This approach is sustainable and inexpensive, as it requires only very small amounts of active matter. These treatments generally resist well to the external aggressions and therefore have many potential uses (operating room, food industry, homes etc.).

Sets of carbon atoms in the sp² hybridization state, for example graphene or graphene oxide flakes, have a proven bactericidal effect. However, it has been shown that the bactericidal effect of the graphene or graphene oxide flakes only comes from the edges of the flakes. The edges of the flakes are so rough that they cause the tearing of the cell membrane of the bacteria and thus the cell death of the bacteria. Thus, the bactericidal power of a surface covered with graphene or graphene oxide flakes will be inversely proportional to the size of the flakes. The smaller the graphene or graphene oxide flakes, the more toxic they are, in particular if swallowed.

In contrast, the sets of carbon atoms in the sp² hybridization state in the continuous layer, in particular the continuous layer graphene, have extremely low toxicity, but no antibacterial character from the basal planar part has been found.

Chemical functionalizations of surfaces to render them antibacterial have been in particular described, generally based on polymers, but these polymers are not transparent (which can be inconvenient for certain applications) and/or do not adapt to all the surfaces to be treated, and/or only confer a bactericidal character to the treated surface, whereas surfaces with bacteriostatic properties are sometimes preferred according to the targeted applications.

The purpose of the invention is to allow the implementation of a method of functionalizing a surface to render it antibacterial which avoids the aforementioned drawbacks.

Thus, an aim of the invention is to provide a method for allowing an antibacterial treatment of any type of surface, whether in terms of physicochemical nature and/or shape, topography.

A further aim of the invention is to provide a method which allows, if necessary, depending on the intended application, an easy and fine tuning of the antibacterial properties of the treated surface, e.g. a bactericidal or bacteriostatic character.

Another purpose of the invention is to provide an antibacterial functionalized surface, the functionalization of which can be transparent to the visible wavelengths.

Thus, according to a first embodiment, the invention relates to a set of carbon atoms in the sp² hybridization state, said set being in contact with:
a plurality of compounds of the following formula (I):

(R-L₁-W)ₙ—V-L₂-X—Y (I),
in which:
n is equal to 1, 2, or 3, in particular 3;
V represents, for all n, —C(H)₃₋ₙ— or:
   when n is equal to 2, —C(H)≡, C being in this case linked to L₂ by a double bond;
   when n is equal to 1, C being in this case linked to L₂ by a triple bond;
R is an aromatic hydrocarbon comprising from 2 to 6 condensed aromatic rings, in particular at each occurrence independently selected from pyrenyl, benzopyrenyl, anthracenyl, chrystenyl, coronenyl, coranulenyl, naphthyl, tetracenyl, pentacenyl, phenathrenyl, triphenylenyl and ovalenyl groups, more particularly from pyrenyl, benzopyrenyl, anthracenyl groups;
L₁ is a C₁-C₁₂ alkanediyl group;
W represents a single bond, an arenediyl group, a heteroarenediyl group or a —O—Ar₁— group wherein Ar₁ is an arene or a heteroarene;
L₂ is a group of formula (A) as follows, -(L₂ₐ)ᵢ-(L_{2b})ⱼ- wherein:
   i and j are independently of each other selected from 0 and 1, with i+j=1 or 2;
   L₂ₐ is a C₁-C₁₂ alkanediyl, C₂-C₁₂ alkenediyl or C₂-C₁₂ alkynediyl group;
   L_{2b} is a arenediyl group, a heteroarenediyl group or a —O—Ar₂— group wherein Ara is an arene or a heteroarene;
X is —C(═O)N—, —C(═O)O— or —C(═O)S— group;
Y is an antibacterial antibody or a lectin;
a plurality of compounds of the following formula (II):

(R-L₁-W)ₙ—V-L₂-X—Z (II),
in which:
n, R, L₁, W, V, L₂ and X are as defined above;
Z is an antibacterial peptide.

Thus, in both formulae (I) and (II), C represents a tetravalent carbon atom in the case wherein V represents —C(H)₃₋ₙ— (—C—, —CH—, or —CH₂— depending on the value of n); a trivalent carbon atom when V represents —C(H)═; or a divalent carbon atom when V represents —C≡.

By “in contact” is meant in particular that the aromatic hydrocarbons (R) of the compounds of formulae (I) and (II) form Pi-stacking bonds with the set of carbon atoms in the sp² hybridization state. These bonds, also called πC-π stacking, are non-covalent bonds, among the strongest of the non-covalent bonds.

According to a particular embodiment, said set of carbon atoms in the sp² hybridization state is selected from graphene, graphene oxide, graphite, carbon nanotubes, fullerenes and fullerites, said set being in particular graphene, more particularly in the form of sheet or flakes.

By “sheet” is meant notably graphene, in particular single layer, two of whose dimensions are greater than 1 mm.

By “flake” is meant notably graphene, in particular with 1 to 10 layers, for example single layer, one or two of whose dimensions are less than or equal to 1 mm, in particular 500 μm, 100 μm or 10 μm.

According to a particular embodiment, the invention relates to a set of carbon atoms in the sp² hybridization state, said set being in contact with:
a plurality of compounds of the following formula (Ia):

(Py-L₁-W)₃—C-L₂-X—Y (Ia),
in which:
Py is a pyrenyl group;
L₁ is a C₁-C₁₂ alkanediyl group;
W represents a single bond, an arenediyl group, a heteroarenediyl group or a —O—Ar₁— group wherein Ar₁ is an arene or a heteroarene;
L₂ is a group of the formula (A) as follows, -(L₂ₐ)ᵢ-(L_{2b})ⱼ- wherein:
   i and j are independently of each other selected from 0 and 1, with i+j=1 or 2;
   L₂ₐ is a C₁-C₁₂ alkanediyl, C₂-C₁₂ alkenediyl or C₂-C₁₂ alkynediyl group;
   L_{2b} is a arenediyl group, a heteroarenediyl group or a —O—Ar₁— group wherein Ara is
   an arene or a heteroarene;
X is —C(═O)N—, —C(═O)O— or —C(═O)S— group;
Y is an antibacterial antibody; and
a plurality of compounds of the following formula (IIa):

(Py-L₁-W)₃—C-L₂-X—Z (IIa),

in which:

Py, L₁, W, L₂ and X are as defined above;

Z is an antibacterial peptide.

According to a particular embodiment, said set of carbon atoms in the sp² hybridization state is supported by a substrate, in particular selected from metals, metal oxides, glasses and polymers.

According to a particular embodiment, the groups R, L₁, W, V, L₂ and optionally X of the compound (I) are identical to those of the compound of formula (II).

According to a particular embodiment, R is the 1-pyrenyl or the 2-pyrenyl, in particular the 1-pyrenyl.

According to a particular embodiment, L₁ is a C₂-C₈ alkanediyl group, in particular the butanediyl.

According to a particular embodiment, W represents a —O—Ar₁— group wherein Ar₁ is an arene or a heteroarene, in particular a benzene, more particularly a para-substituted benzene.

According to a particular embodiment, i and j are both equal to 1.

According to a particular embodiment, L₂ₐ is a C₂-C₁₂ alkynediyl group, in particular —C≡C—.

According to a particular embodiment, L_{2b} is a arenediyl group, in particular a benzenediyl, more particularly a para-substituted benzenediyl.

According to a particular embodiment, R, L₁, W, V, L₂ are as defined by the following formula:

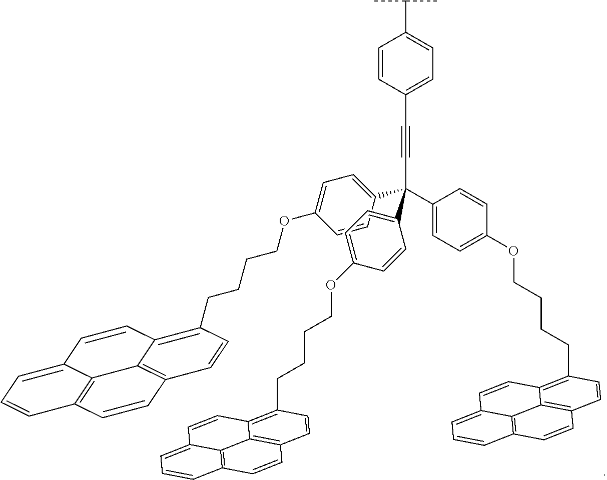

According to a particular embodiment, Y is selected from the anti-Escherichia coli antibodies, the anti-gram positive bacteria antibodies, the anti-gram negative bacteria antibodies, Y being in particular an anti-Escherichia coli antibody.

The anti-Escherichia coli antibodies are well known to the person skilled in the art. For example, polyclonal antibodies, in particular from goat or from rabbit, in particular those recognizing numerous antigenic “O” and “K” serotypes of Escherichia coli, in particular those marketed by LifeSpan BioSciences (e.g. under the reference LS-058854) or by Abcam (e.g. under the reference ab137967).

The antibodies may also be anti-Escherichia coli antibodies, preferably monoclonal, in particular from mice, specifically directed against particular strains of E. coli, for example particularly pathogenic strains such as the strain 0157. This is in particular the case for the anti E Coli 0157 antibodies marketed by MyBioSource (for example under the reference MBS568193).

The anti-gram positive bacteria antibodies and the anti-gram negative bacteria antibodies are also well known to the person skilled in the art. These may include monoclonal antibodies directed against the endotoxins of the Gram-negative bacteria, in particular those marketed by ThermoFisher Scientific (for example under the reference MA1-10685).

According to a particular embodiment, Y is selected from the lectins, in particular those binding to sugars of the bacterial walls, more particularly the sugars present on the wall of the Gram-positive bacteria.

According to a particular embodiment, Y is the concanavalin A. This compound is in particular marketed by Sigma-Aldrich (for example under the reference C5275).

According to a particular embodiment, Z is selected from cecropins, defensins, magainins and dermaseptins, Z being in particular a cecropin.

According to a more particular embodiment, Z is selected from the cecropins, in particular cecropin A (SEQ ID NO:1), cecropin B (SEQ ID NO:2), cecropin P1 (SEQ ID NO:3), cecropin 1 (SEQ ID NO:4) and Bombyx mori cecropin (SEQ ID NO:5), the defensins, in particular defensin 6 (SEQ ID NO:6) and defensin 5 (SEQ ID NO:7), the magainins, in particular magainin B2 (SEQ ID NO:8), and magainin R2 (SEQ ID NO:9) and the dermaseptins, in particular dermaseptin H3 (SEQ ID NO:10) and dermaseptin 51 (SEQ ID NO:11).

The cecropins A, B and P1 are in particular marketed by Sigma Aldrich.

According to a particular embodiment, the density of compounds of formulae (I) and (II) on the surface of the set of carbon atoms in the sp² hybridization state is comprised from about 1 of these compounds per 300 nm² area to about 1 of these compounds per 2 nm² area, in particular about 1 compound per about 2.7 nm² area.

According to a particular embodiment, the molar ratio of the amount of compounds of formula (I) to that of compounds of formula (II) is comprised from 0.01 to 100, in particular from 0.1 to 10, in particular from 0.33 to 1.5, more particularly from 0.33 to 0.66 or from 0.66 to 1.5, for example about 0.5 or about 1.0.

According to a more particular embodiment, the set of carbon atoms in the sp² hybridization state is graphene in the form of sheet, the molar ratio of the amount of compounds of formula (I) to that of compounds of formula (II) being comprised from 0.66 to 1.5, for example about 1.0.

According to a more particular embodiment, the set of carbon atoms in the sp² hybridization state is graphene in the form of flakes, the molar ratio of the amount of compounds of formula (I) to that of compounds of formula (II) being comprised from 0.33 to 0.66, for example about 0.5.

According to another embodiment, the invention also relates to a method for preparing a functionalized set of carbon atoms in the sp² hybridization state as defined above, comprising the following steps:

(i) contacting a set of carbon atoms in the sp² hybridization state with a compound of the following formula (III):

(R-L₁-W)n-V-L₂-X-Q (III),
in which:
R, L₁, W, V and L₂ are defined as before;
X-Q is a group selected from —C(═O)O—N-succinimidyl, —C(═O)-halide, in particular —C(═O)—Cl, —C(═O)—N₃, —C(═O)—O—N-imidazolyl, —C(═O)—O—C(═O)—R′, with R′ being a linear, branched or cyclic C₁-C₁₂ alkyl, —C(═O)—O-catecholborane, —C(═O)—O-benzotriazole, in particular —C(═O)O—N-succinimidyl,
to obtain a set of carbon atoms in the activated sp² hybridization state;
   (ii) contacting the set of carbon atoms in the activated sp² hybridization state as obtained in the previous step with a composition comprising an antibacterial antibody or a lectin and an antibacterial peptide, to obtain a functionalized set of carbon atoms in the sp² hybridization state.

Alternatively, the contacting of the step (ii) may be sequential, i.e. comprising a first contacting with a composition comprising an antibacterial antibody or a lectin and then a second contacting with a composition comprising an antibacterial peptide, or a first contacting with a composition comprising an antibacterial peptide and then a second contacting with a composition comprising an antibacterial antibody or a lectin.

This method allows the antibacterial treatment of any set of carbon atoms in the sp² hybridization state, in particular graphene, especially the graphene of the biosensors of the graphene.

All of the embodiments defined above with respect to the (functionalized) set of carbon atoms in the sp² hybridization state also apply herein, alone or in combination.

According to another embodiment, the invention also relates to the use of a set of carbon atoms in the sp² hybridization state as defined above, for rendering a surface antibacterial, in particular bactericidal or bacteriostatic.

All of the embodiments defined above with respect to the set of carbon atoms in the sp² hybridization state also apply herein, alone or in combination.

According to another embodiment, the invention also relates to a material comprising a substrate all or part of the surface of which covered with a functionalized set of carbon atoms in the sp² hybridization state (i.e., in contact with compounds of formulae (I) and (II)) as defined above.

All of the embodiments defined above with respect to the set of carbon atoms in the sp² hybridization state also apply herein, alone or in combination.

According to a particular embodiment, the substrate is selected from metals, metal oxides, glasses and polymers.

According to another embodiment, the invention also relates to a method for preparing a material as defined above, comprising the following steps:

(i) contacting a set of carbon atoms in the sp² hybridization state with a compound of the following formula (III):

(R-L₁-W)ₙ—V-L₂-X-Q (III),
in which:
R, L₁, W, V, and L₂ are as defined above;
X-Q is a group selected from —C(═O)O—N-succinimidyl, —C(═O)-halide, in particular —C(═O)—Cl, —C(═O)—N₃, —C(═O)—O—N-imidazolyl, —C(═O)—O—C(═O)—R′, with R′ being a linear, branched or cyclic C₁-C₁₂ alkyl, —C(═O)—O-catecholborane, —C(═O)—O-benzotriazole, in particular —C(═O)O—N-succinimidyl,
to obtain a set of carbon atoms in the activated sp² hybridization state;
   (ii) contacting the set of carbon atoms in the activated sp² hybridization state as obtained in the previous step with a composition comprising an antibacterial antibody or a lectin and an antibacterial peptide, to obtain a functionalized set of carbon atoms in the sp² hybridization state;
   (iii) depositing the functionalized set of carbon atoms in the sp² hybridization state as obtained in the previous step on the surface of a support to obtain said material.

All of the embodiments defined above with respect to the set of carbon atoms in the sp² hybridization state and to a method also apply herein, alone or in combination.

According to a particular embodiment, the contacting in the step (i) is carried out using a solution of the compound of formula (III) in a solvent.

According to a more particular embodiment, said solvent is selected from the aprotic apolar solvents, in particular tetrahydrofuran, n-hexane, cyclohexane, 1,4-dioxane, toluene, diethyl ether, ethyl acetate, dichloromethane, in particular tetrahydrofuran.

According to a more particular embodiment, the compound of formula (III) is present in the solution at a concentration comprised from 1 nM to 100 for example at about 104.

According to a particular embodiment, the contacting in the step (i) is done for a period of time of from 10 seconds to 300 seconds, for example about 60 seconds.

According to a particular embodiment, the step (i) is followed, prior to the step (ii) by a step of washing the set of carbon atoms in the activated sp² hybridization state, in particular with water.

According to a particular embodiment, the contacting in the step (ii) is carried out using a solution of the antibacterial antibody or of the lectin and the antibacterial peptide in a solvent.

According to a more particular embodiment, said solvent is selected from the phosphate buffered saline solutions, in particular the PBS 1X.

In a more particular embodiment, the antibacterial antibody or the lectin and the antibacterial peptide are present in the solution at a total concentration comprised from 10 pM to 10 μM, for example at about 10 nM.

According to a particular embodiment, the contacting in the step (ii) is carried out for a period of 10 minutes to 100 minutes, for example about 30 minutes.

According to a particular embodiment, the step (ii) is followed, prior to the step (iii), by a step of washing the set of carbon atoms in the functionalized sp² hybridization state, in particular using a phosphate buffered saline solution, in particular the PBS 1X.

According to a particular embodiment, the deposition of the functionalized set of carbon atoms in the sp² hybridization state of the step (iii) is done by a transfer from a substrate to said surface, in particular from a copper substrate, in particular by wet transfer, for example according to Won Suk et al. (ACS Nano 2011, 5(9), 6916-6924), or by dry transfer, for example according to Vaziri et al. (PDMS-supported graphene transfer using intermediary polymer layers, 2014 44th European Solid State Device Research Conference (ESSDERC), 309-312) or Caldwell et al. (ACS Nano 2010, 4(2), 1108-1114).

As an example for the graphene, during a wet transfer, the copper layer used to grow the graphene can in particular be dissolved on the surface of a etching bath of the copper (liquid) and then the graphene is contacted with the surface of said support to obtain said material.

Also as an example for the graphene, during a dry transfer, the graphene can in particular be detached from the copper growth substrate by adding a polymer (e.g. the parylene PDMS), then the polymer+graphene set is applied to the desired surface by mechanical action (mechanical peeling). The polymer is then removed.

According to another embodiment, the invention relates to a method for preparing a material as defined above, comprising the following steps:

(i) contacting a set of carbon atoms in the sp² hybridization state carried on the surface of a material with a compound of the following formula (III):

(R-L₁-W)n-V-L₂-X-Q (III),
in which:
R, L₁, W, V, and L₂ are as defined above;
X-Q is a group selected from —C(═O)O—N-succinimidyl, —C(═O)-halide, in particular —C(═O)—Cl, —C(═O)—N₃, —C(═O)—O—N-imidazolyl, —C(═O)—O—C(═O)—R′, with R′ being a linear, branched or cyclic C₁-C₁₂ alkyl, —C(═O)—O-catecholborane, —C(═O)—O-benzotriazole, in particular —C(═O)O—N-succinimidyl,
to obtain an activated material;
   (ii) contacting the activated material as obtained in the previous step with a composition comprising an antibacterial antibody or a lectin and an antibacterial peptide, to obtain said material, functionalized.

This method thus allows to treat any material carrying a set of carbon atoms in the sp² hybridization state, for example graphene, and the devices comprising said material.

For example, this method allows the antibacterial treatment of graphene biosensors.

All of the embodiments defined above with respect to the set of carbon atoms in the sp² hybridization state and to a method also apply herein, alone or in combination.

According to a particular embodiment, the set of carbon atoms in the sp² hybridization state carried by the surface of the material is previously deposited on said surface by means of any technique well known to the person skilled in the art.

In the case of the graphene in the form of sheet, said sheet may in particular be obtained by any technique known to the person skilled in the art, for example by chemical vapor deposition (CVD) transfer, notably on copper substrate, in particular as described by Li et al. (Journal of the American Chemical Society 2011, 133, 2816-2819), then by transferring the resulting sheet to said surface by any technique known to the person skilled in the art, for example by liquid transfer, in particular as described by Reina et al. (Journal of the American Chemical Society 2011, 133, 17614-17617).

### Definitions

As used in this description, the term “about” refers to a range of values within ±10% of a specific value. For example, the term “about 20” comprises the values of 20±10%, i.e., the values of 18 to 22.

For the purposes of this description, the percentages refer to percentages by mass in relation to the total mass of the formulation, unless otherwise stated.

As understood here, the value ranges in the form of “x-y” or “from x to y” or “between x and y” include the bounds x and y as well as the integers between these bounds. For example, “1-5”, or “from 1 to 5” or “between 1 and 5” refer to the integers 1, 2, 3, 4 and 5. The preferred embodiments include each individual integer in the value range, as well as any sub-combination of those integers. For example, the preferred values for “1-5” may comprise the integers 1, 2, 3, 4, 5, 1-2, 1-3, 1-4, 1-5, 2-3, 2-4, 2-5, etc.

As used herein, the term “alkyl” means a linear or branched chain alkyl group having the number of carbon atoms indicated before said term, in particular 1 to 8 carbon atoms, such as methyl, ethyl, propyl, isopropyl, butyl, isobutyl, sec-butyl, tert-butyl, pentyl, isoamyl, neopentyl, 1-ethylpropyl, 3-methylpentyl, 2,2-dimethylbutyl, 2,3-dimethylbutyl, hexyl, octyl, etc. Thus, an expression such as “C1-C4 alkyl” designates an alkyl radical containing from 1 to 4 carbon atoms. The same applies to the term “alkane”.

As used herein, the term “arene” refers to a substituted or unsubstituted mono- or bicyclic aromatic hydrocarbon ring system having 6 to 10 carbon atoms in the ring. The examples include the benzene and the naphthalene. The preferred arenes include the unsubstituted or substituted benzene and naphthalene. The definition of “arene” includes condensed ring systems, including, for example, ring systems in which an aromatic ring is condensed to a cycloalkyl ring. The examples of such condensed ring systems comprise, for example, the indane, the indene and the tetrahydronaphthalene.

As used herein, the term “heteroarene” refers to an aromatic ring system containing 5 to 10 carbon atoms in which one or more carbon atoms of the ring are replaced by at least one heteroatom such as —O—, —N— or —S—. The examples of heteroarenes comprise pyrrole, furan, thiophene, pyrazole, imidazole, thiazole, isothiazole, isoxazole, oxazole, oxathiol, oxadiazole, triazole, oxatriazole, furazane, tetrazole, pyridine, pyrazine, pyrimidine, pyridazine, triazine, indole, isoindole, indazole, benzofuran, isobenzofuran, purine, quinazoline, quinoline, isoquinoline, benzoimidazole, benzothiazole, benzothiophene, thianaphthene, benzoxazole, benzisoxazole, cinnoline, phthalazine, naphthyridine and quinoxaline. The definition of “heteroarene” includes the condensed ring systems, including, for example, the ring systems in which an aromatic ring is condensed to a heterocycloalkyl ring. The examples of such condensed ring systems comprise, for example, phthalamide, phthalic anhydride, indoline, isoindoline, tetrahydroisoquinoline, chroman, isochroman, chromen and isochromene.

TABLE 1

| | |
|---|---|
| SEQ ID NO: 1 | Lys Trp Lys Leu Phe Lys Lys Ile Glu |
| | Lys Val Gly Gln Asn Ile Arg Asp Gly |
| | Ile Ile Lys Ala Gly Pro Ala Val Ala |
| | Val Val Gly Gln Ala Thr Gln Ile Ala |
| | Lys |
| SEQ ID NO: 2 | Lys Trp Lys Val Phe Lys Lys Ile Glu |
| | Lys Met Gly Arg Asn Ile Arg Asn Gly |
| | Ile Val Lys Ala Gly Pro Ala Ile Ala |
| | Val Leu Gly Glu Ala Lys Ala Leu |
| SEQ ID NO: 3 | Ser Trp Leu Ser Lys Thr Ala Lys Lys |
| | Leu Glu Asn Ser Ala Lys Lys rg Ile |
| | Ser Glu Gly Ile Ala Ile Ala Ile Gln |
| | Gly Gly Pro Arg |
| SEQ ID NO: 4 | Met Asn Phe Asn Lys Val Phe Ile Leu |
| | Val Ala Ile Val Ile Ala Ile Phe Ala |
| | Gly Gln Thr Glu Ala Gly Trp Leu Lys |
| | Lys Ile Gly Lys Lys Ile Glu Arg Val |
| | Gly Gln His Thr Arg Asp Ala Thr Ile |
| | Gln Thr Ile Ala Val Ala Gln Gln Ala |
| | Ala Asn Val Ala Ala Thr Ala Arg Gly |
| SEQ ID NO: 5 | Arg Trp Lys Ile Phe Lys Lys Ile Glu |
| | Lys Val Gly Gln Asn Ile Arg Asp Gly |
| | Ile Val Lys Ala Gly Pro Ala Val Ala |
| | Val Val Gly Gln Ala Ala Thr Ile |
| SEQ ID NO: 6 | Met Arg Thr Leu Thr Ile Leu Thr Ala |
| | Val Leu Leu Val Ala Leu Gln Ala Lys |
| | Ala Glu Pro Leu Gln Ala Glu Asp Asp |
| | Pro Leu Gln Ala Lys Ala Tyr Glu Ala |
| | Asp Ala Gln Glu Gln Arg Gly Ala Asn |
| | Asp Gln Asp Phe Ala Val Ser Phe Ala |
| | Glu Asp Ala Ser Ser Ser Leu Arg Ala |
| | Leu Gly Ser Thr Arg Ala Phe Thr Cys |
| | His Cys Arg Arg Ser Cys Tyr Ser Thr |
| | Glu Tyr Ser Tyr Gly Thr Cys Thr Val |
| | Met Gly Ile Asn His Arg Phe Cys Cys |
| | Leu |
| SEQ ID NO: 7 | Met Arg Thr Ile Ala Ile Leu Ala Ala |
| | Ile Leu Leu Val Ala Leu Gln Ala Gln |
| | Ala Glu Ser Leu Gln Glu Arg Ala Asp |
| | Glu Ala Thr Thr Gln Lys Gln Ser Gly |
| | Glu Asp Asn Gln Asp Leu Ala Ile Ser |
| | Phe Ala Gly Asn Gly Leu Ser Ala Leu |
| | Arg Thr Ser Gly Ser Gln Ala Arg Ala |
| | Thr Cys Tyr Cys Arg Thr Gly Arg Cys |
| | Ala Thr Arg Glu Ser Leu Ser Gly Val |
| | Cys Glu Ile Ser Gly Arg Leu Tyr Arg |
| | Leu Cys Cys Arg |
| SEQ ID NO: 8 | Gly Ile Gly Lys Phe Leu His Ser Ala |
| | Gly Lys Phe Gly Lys Ala Phe Leu Gly |
| | Glu Val Met Lys Ser |
| SEQ ID NO: 9 | Gly Ile Lys Glu Phe Ala His Ser Leu |
| | Gly Lys Phe Gly Lys Ala Phe Val Gly |
| | Gly Ile Leu Asn Gln |
| SEQ ID NO: 10 | Met Ala Phe Leu Lys Lys Ser Leu Phe |
| | Leu Val Leu Phe Leu Gly Met Val Ser |
| | Leu Ser Ile Cys Glu Glu Glu Lys Arg |
| | Glu Asn Glu Asp Glu Glu Leu Gln Glu |
| | Asp Asp Glu Gln Ser Glu Met Lys Arg |
| | Gly Leu Trp Ser Thr Ile Lys Asn Val |
| | Gly Lys Glu Ala Ala Ile Ala Ala Gly |
| | Lys Ala Ala Leu Gly Ala Leu |
| SEQ ID NO: 11 | Met Asp Ile Leu Lys Lys Ser Leu Phe |
| | Leu Val Leu Phe Leu Gly Leu Val Ser |
| | Leu Ser Ile Cys Glu Glu Glu Lys Arg |
| | Glu Asn Glu Asp Glu Glu Lys Gln Glu |
| | Asp Asp Glu Gln Ser Glu Met Lys Arg |
| | Ala Leu Trp Lys Thr Met Leu Lys Lys |
| | Leu Gly Thr Met Ala Leu His Ala Gly |
| | Lys Ala Ala Leu Gly Ala Ala Ala Asp |
| | Thr Ile Ser Gln Gly Thr Gln |

### FIGURES

FIG. 1 shows optical images related to the growth of E. coli versus time, according to Example 2, with a surface such that the ratio of A:B is 0.35:0.65 (7:13).

FIG. 2 shows optical images related to the growth of E. coli versus time, according to Example 2, with a surface such that the ratio of A:B is 0.5:0.5 (1:1).

### EXAMPLES

### Example 1: Preparation of a Set of Carbon Atoms in the Sp² Hybridization State Functionalized with an Antibacterial Antibody or a Lectin and an Antibacterial Peptide

A set of carbon atoms in the sp² hybridization state, for example a graphene layer, is synthesized according to any technique well known to the person skilled in the art, in particular by CVD, then optionally deposited on a surface to be treated by any technique well known to the person skilled in the art, in particular by a liquid transfer method.

The graphene layer is soaked in a 1 μM solution in the tetrahydrofuran (THF) of a tripod of the following formula (as described for example by Mann et al., Angewandte Chemie International Edition 2013, 52, 3177-3180):

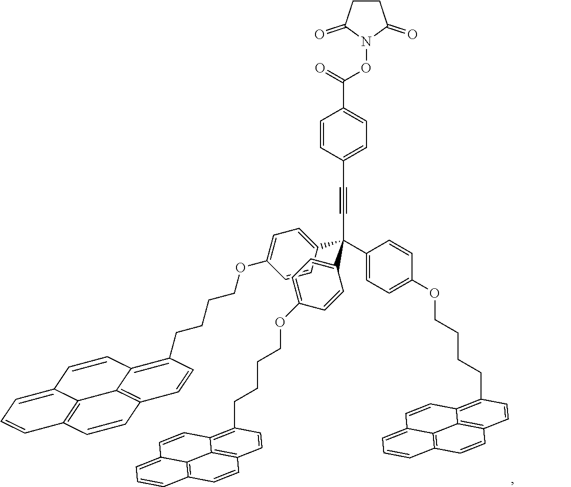

and this, for one minute. The sample is then soaked in water in order to wash off the excess of THF. This results in a monolayer of tripods on the surface of the graphene with a density of about 1 tripod molecule per 2.7 nm².

The resulting sample is incubated in a solution of PBS 1X. Anti-Escherichia coli antibodies (which have the ability to specifically bind Escherichia coli type bacteria) such as the LifeSpan BioSciences LS-058854, Abcam ab137967 or MyBioSource MBS568193 are then added to this solution, as well as molecules of cecropin (which have the particularity of creating holes in the cell membrane of bacteria and causing their cell death), such as cecropin A, cecropin B or cecropin P1 marketed by Sigma Aldrich (under the reference C6830 for cecropin A), in precise molar proportions, which are in particular discussed in the following example.

The tripod is designed in such a way that the N-Hydroxysuccinimide ester present on the top of the molecule will engage covalently bonds to any type of peptide or protein.

Thus, by injecting the antibody solution: cecropin into a stoichiometric A:B mixture, these molecules will covalently attach themselves one by one to the top of a tripod. After 30 minutes of incubation, there is an A:B ratio of antibodies and cecropin on the surface of the graphene.

Following the deposition of these molecules, the molecules ungrafted to a tripod are rinsed with a fresh solution of PBS1X (buffer solution) for 5 min.

### Example 2: Evaluation of the Antibacterial Character of a Functionalized Set of Carbon Atoms in the Sp² Hybridization State According to the Invention

Protocol

A graphene layer e.g. produced by CVD (Li et al., Journal of the American Chemical Society 2011, 133, 2816-2819) is deposited (e.g. according to Reina et al. Journal of the American Chemical Society 2011, 133, 17614-17617) on the surface of a microscope glass slide.

This graphene layer is functionalized according to the invention, in particular according to Example 1. Then, bacteria of Escherichia coli K12 type, which have been modified with a plasmid that allows them to synthesize the fluorescent protein Green Fluorescent Protein in addition to their metabolism (which allows the bacteria to be observed by fluorescence microscopy), are incubated on the surface for 30 min.

The bacteria that are not attached to the surface are then rinsed with PBS1X solution for 5 min. Then a nutrient solution for bacteria (Luria Bertani solution, also known as LB culture medium) is poured in. The temperature is maintained at 37° C. The bacteria attached to the surface of the graphene are thus, apart from the antibacterial surface, in conditions of exponential growth and proliferation.

Results

A control not part of the invention (surface with an A:B (antibody:antimicrobial peptide) ratio of 1:0) shows an exponential growth of the bacteria on the surface which starts to form a biofilm.

A:B Ratio from 0.25:0.75 to 0.40:0.60, in Particular 0.35:0.65 (7:13)

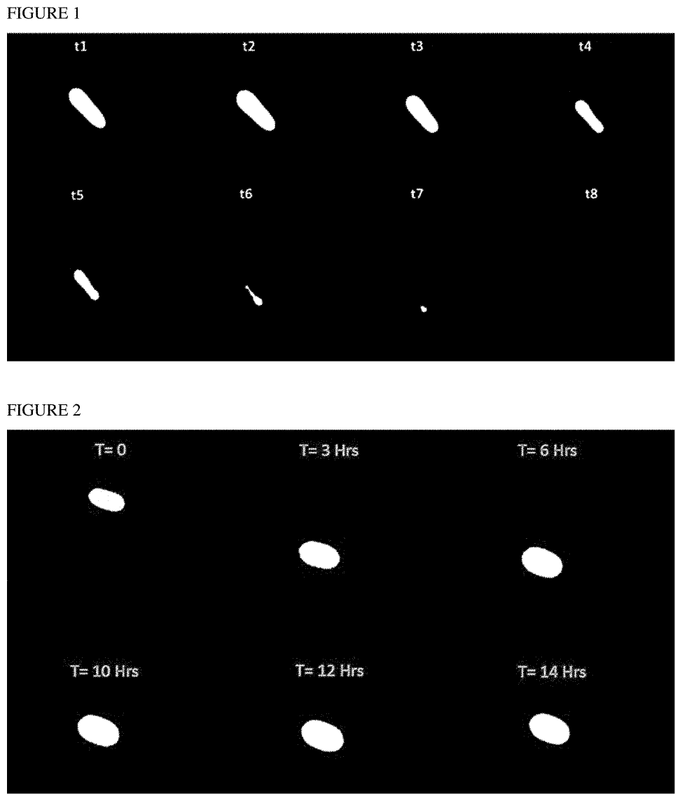

With such a ratio, it is observed that the functionalized layer of the invention causes not only the arrest of the growth of the bacteria on the surface but also their cell death. This is confirmed by the analysis of the fluorescence signal over time (FIG. 1). This signal decreases, which means that the cell membrane is damaged to such an extent that the GFP proteins leave the bacterium, which is therefore considered dead (or lysed).

Thus, such a ratio allows to obtain a bactericidal surface.

A:B Ratio Comprised from 0.40:0.60 to 0.60:0.40, in Particular 0.5:0.5 (1:1) With such a ratio, it is observed that the functionalized layer of the invention causes the bacteria to stop growing on the surface but the bacteria are not killed. Indeed, the signal related to the total GFP fluorescence intensity is constant over time (FIG. 2).

Thus, with this ratio, the layer still has a property of limiting the biofilm growth but the bacteria are no longer killed. It is therefore a bacteriostatic effect.

1. A set of carbon atoms in the sp² hybridization state, said set being in contact with:
a plurality of compounds of the following formula (I):
(R-L₁-W)ₙ—V-L₂-X—Y (I),
in which:
n is equal to 1, 2, or 3, in particular 3;
V represents, for all n, —C(H)₃₋ₙ— or:
when n is equal to 2, —C(H)═, C being in this case linked to L₂ by a double bond;
when n is equal to 1, —C≡, C being in this case linked to L₂ by a triple bond;
R is an aromatic hydrocarbon comprising from 2 to 6 condensed aromatic rings;
L₁ is a C₁-C₁₂ alkanediyl group;
W represents a single bond, an arenediyl group, a heteroarenediyl group or a —O—Ar₁— group wherein Ar₁ is an arene or a heteroarene;
L₂ is a group of the following formula (A) -(L₂ₐ)ᵢ-(L_{2b})ⱼ- wherein:
i and j are independently of each other selected from 0 and 1, with i+j=1 or 2;
L₂ₐ is a C₁-C₁₂ alkanediyl, C₂-C₁₂ alkenediyl or C₂-C₁₂ alkynediyl group;
L_{2b} is a arenediyl group, a heteroarenediyl group or a —O—Ar₂— group wherein Ar₂ is an arene or a heteroarene;
X is —C(═O)N—, —C(═O)O— or —C(═O)S— group;
Y is an antibacterial antibody or a lectin;
and
a plurality of compounds of the following formula (II):
(R-L₁-W)ₙ—V-L₂-X—Z (II),
in which:
R, L₁, W, V, L₂ and X are as defined above;
Z is an antibacterial peptide.

2. The set according to claim 1, wherein said set of carbon atoms in the sp² hybridization state is selected from graphene, graphene oxide, graphite, carbon nanotubes, fullerenes and fullerites.

3. The set according to claim 1, wherein:
R is the 1-pyrenyl or the 2-pyrenyl;
L₁ is a C₂-C₈ alkanediyl group; and/or
W represents a —O—Ar₁— group wherein Ar₁ is an arene or a heteroarene; and/or
i and j are equal to 1; and/or
L₂ₐ is a C₂-C₁₂ alkynediyl group; and/or
L_{2b} is an arenediyl group; and/or
R, L₁, W, V and G are as defined by the following formula:

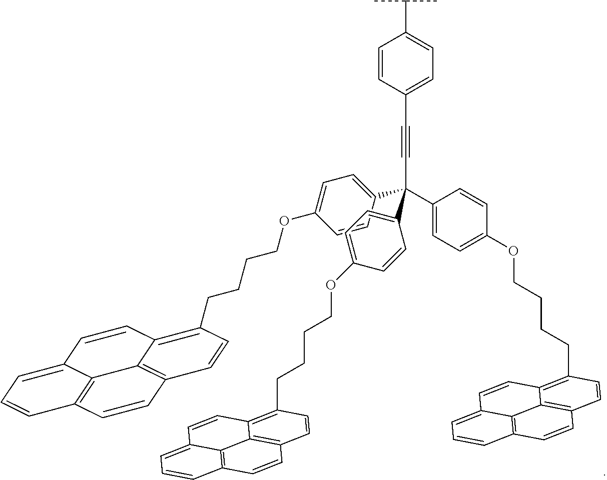

4. The set according to claim 1, wherein:
Y is selected from the anti-Escherichia coli antibodies, the anti-gram positive bacteria antibodies, the anti-gram negative bacteria antibodies and the concanavalin A; and/or
Z is selected from cecropins, defensins, magainins and dermaseptins.

5. The set according to claim 1, wherein the density of compounds of formulae (I) and (II) on the surface of the set of carbon atoms in the sp² hybridization state is comprised from about 1 of these compounds per 300 nm² area to about 1 of these compounds per 2 nm² area.

6. The set according to claim 1, wherein:
the molar ratio of the amount of compounds of formula (I) to that of compounds of formula (II) is comprised from 0.01 to 100;
the set of carbon atoms in the sp² hybridization state is graphene in the form of sheet, the molar ratio of the amount of compounds of formula (I) to that of compounds of formula (II) being comprised from 0.66 to 1.5, for example about 1.0; and/or
the set of carbon atoms in the sp² hybridization state is graphene in the form of flakes, the molar ratio of the amount of compounds of formula (I) to that of compounds of formula (II) being comprised from 0.33 to 0.66, for example about 0.5.

7. A method of preparing a set of carbon atoms in the sp² hybridization state according to claim 1, comprising the following steps:
(i) contacting a set of carbon atoms in the sp² hybridization state with a compound of the following formula (III):
(R-L₁-W)ₙ—V-L₂-X-Q (III),
in which:
R, L₁, W, V and L₂ are as defined in claim 1;
X-Q is a group selected from —C(═O)O—N-succinimidyl, —C(═O)-halide —C(═O)—N₃, —C(═O)—O—N-imidazolyl, —C(═O)—O—C(═O)—R′, with R′ being a linear, branched or cyclic C₁-C₁₂ alkyl, —C(═O)—O-catecholborane, —C(═O)—O-benzotriazole,
to obtain a set of carbon atoms in the activated sp² hybridization state;
(ii) contacting the set of carbon atoms in the activated sp² hybridization state as obtained in the previous step with a composition comprising an antibacterial antibody or a lectin and an antibacterial peptide, to obtain a functionalized set of carbon atoms in the sp² hybridization state.

8. A surface antibacterial composition comprising the set of carbon atoms in the sp² hybridization state according to claim 1.

9. A material comprising a substrate all or part of the surface of which is covered with a set of carbon atoms in the sp² hybridization state in contact with compounds of formulae (I) and (II) according to claim 1.

10. The material according to claim 9, wherein said set of carbon atoms in the sp² hybridization state is supported by a substrate.

11. A method for preparing a material according to claim 9, comprising the following steps:
(i) contacting a set of carbon atoms in the sp² hybridization state with a compound of the following formula (III):
(R-L₁-W)ₙ—V-L₂-X-Q (III),
in which:
X-Q is a group selected from —C(═O)O—N-succinimidyl, —C(═O)-halide —C(═O)—N₃, —C(═O)—O—N-imidazolyl, —C(═O)—O—C(═O)—R′, with R′ being a linear, branched or cyclic C₁-C₁₂ alkyl, —C(═O)—O-catecholborane, —C(═O)—O-benzotriazole,
to obtain a set of carbon atoms in the activated sp² hybridization state;
(ii) contacting the set of carbon atoms in the activated sp² hybridization state as obtained in the previous step with a composition comprising an antibacterial antibody or a lectin and an antibacterial peptide, to obtain a functionalized set of carbon atoms in the sp² hybridization state;
(iii) depositing the functionalized set of carbon atoms in the sp² hybridization state as obtained in the previous step on the surface of a support to obtain said material.

12. A method for preparing a material according to claim 9, comprising the following steps:
(i) contacting a set of carbon atoms in the sp² hybridization state carried on the surface of a material with a compound of the following formula (III):
(R-L₁-W)ₙ—V-L₂-X-Q (III),
in which:
X-Q is a group selected from —O—N-succinimidyl, -halide, —N₃, —O—N-imidazolyl, —O—C(═O)—R′, with R′ being a linear, branched or cyclic C₁-C₁₂ alkyl, —O-catecholborane, —O-benzotriazole,
to obtain an activated material;
(ii) contacting the activated material as obtained in the previous step with a composition comprising an antibacterial antibody or a lectin and an antibacterial peptide, to obtain said material, functionalized.


SEQUENCE LISTING (11 sequences)

SEQ ID NO: 1 - protein, 37 aa, Hyalophora cecropia
KWKLFKKIEKVGQNIRDGIIKAGPAVAVVGQATQIAK

SEQ ID NO: 2 - protein, 35 aa, Hyalophora cecropia
KWKVFKKIEKMGRNIRNGIVKAGPAIAVLGEAKAL

SEQ ID NO: 3 - protein, 31 aa, Hyalophora cecropia
SWLSKTAKKLENSAKKRISEGIAIAIQGGPR

SEQ ID NO: 4 - protein, 63 aa, Ceratitis capitata
MNFNKVFILVAIVIAIFAGQTEAGWLKKIGKKIERVGQHTRDATIQTIAVAQQAANVAATARG

SEQ ID NO: 5 - protein, 35 aa, Bombyx mori
RWKIFKKIEKVGQNIRDGIVKAGPAVAVVGQAATI

SEQ ID NO: 6 - protein, 100 aa, Homo sapiens
MRTLTILTAVLLVALQAKAEPLQAEDDPLQAKAYEADAQEQRGANDQDFAVSFAEDASSSLRALGSTRAFTCHCRRSCYSTEYSYGTCTVMGINHRFCCL

SEQ ID NO: 7 - protein, 94 aa, Homo sapiens
MRTIAILAAILLVALQAQAESLQERADEATTQKQSGEDNQDLAISFAGNGLSALRTSGSQARATCYCRTGRCATRESLSGVCEISGRLYRLCCR

SEQ ID NO: 8 - protein, 23 aa, Xenopus borealis
GIGKFLHSAGKFGKAFLGEVMKS

SEQ ID NO: 9 - protein, 23 aa, Xenopus ruwenzoriensis
GIKEFAHSLGKFGKAFVGGILNQ

SEQ ID NO: 10 - protein, 70 aa, Phyllomedusa hypochondrialis
MAFLKKSLFLVLFLGMVSLSICEEEKRENEDEELQEDDEQSEMKRGLWSTIKNVGKEAAIAAGKAALGAL

SEQ ID NO: 11 - protein, 79 aa, Phyllomedusa sauvagei
MDILKKSLFLVLFLGLVSLSICEEEKRENEDEEKQEDDEQSEMKRALWKTMLKKLGTMALHAGKAALGAAADTISQGTQ